(19) United States
(12) Patent Application Publication
(10) Pub. No.: US 2018/0180612 A1
(43) Pub. Date: Jun. 28, 2018

(54) MONOCYTE INTEGRIN BASED MICROFLUIDIC ASSAY FOR EVALUATING CORONARY DISEASES

(71) Applicant: THE REGENTS OF THE UNIVERSITY OF CALIFORNIA, Oakland, CA (US)

(72) Inventors: Scott Simon, Davis, CA (US); Greg Foster, Davis, CA (US); Mahshid Mohammadi, Davis, CA (US)

(73) Assignee: THE REGENTS OF THE UNIVERSITY OF CALIFORNIA, Oakland, CA (US)

(21) Appl. No.: 15/846,628

(22) Filed: Dec. 19, 2017

Related U.S. Application Data
(63) Continuation of application No. PCT/US2016/038416, filed on Jun. 20, 2016.
(60) Provisional application No. 62/182,512, filed on Jun. 20, 2015.

(51) Int. Cl. G01N 33/569; B01L 3/00; G01N 33/58; G01N 21/64

(52) U.S. Cl. CPC G01N 33/56972; G01N 2021/6441; B01L 3/50273; G01N 33/582; G01N 21/6428; G01N 2333/70542; G01N 2333/70553; B01L 2400/0487; B01L 2300/0819; B01L 2300/0883; B01L 2300/16; B01L 2200/0647; B01L 2400/084; B01L 2200/10; G01N 2800/324; B01L 3/502715

(57) ABSTRACT

A diagnostic system for assessing cardiovascular health is provided that incorporates a microfluidic platform and sensors that capture inflammatory monocytes. The portable microfluidic platform shears activated monocytes in a small volume of blood (˜50 μL) over a glass substrate that mimics the stress and molecular constituents of an inflamed artery. The sensor utilizes CD11c antibodies and/or VLA-4 ligands to capture cells. The device captures a subset of inflammatory subset of activated white blood cells that play a critical role in the progression of cardiovascular disease and whose numbers in the blood and the efficiency of capture directly correlate with risk and pathogenesis of cardiovascular disease. The risk of future cardiac events can be assessed. The system can facilitate early detection of cardiovascular disease and can guide risk factor modification and therapy following treatment.

### CROSS-REFERENCE TO RELATED APPLICATIONS

This application is a 35 U.S.C. § 111(a) continuation of PCT international application number PCT/US2016/038416 filed on Jun. 20, 2016, incorporated herein by reference in its entirety, which claims priority to, and the benefit of, U.S. provisional patent application Ser. No. 62/182,512 filed on Jun. 20, 2015, incorporated herein by reference in its entirety. Priority is claimed to each of the foregoing applications.

The above-referenced PCT international application was published as PCT International Publication No. WO 2016/209775 on Dec. 29, 2016, which publication is incorporated herein by reference in its entirety.

### STATEMENT REGARDING FEDERALLY SPONSORED RESEARCH OR DEVELOPMENT

This invention was made with Government support under 082689, awarded by the National Institutes of Health. The Government has certain rights in the invention.

### INCORPORATION-BY-REFERENCE OF COMPUTER PROGRAM APPENDIX

Not Applicable

### BACKGROUND

### 1. Technical Field

The present technology pertains generally to diagnostic devices and methods and more particularly to a clinical risk prediction tool that utilizes a microfluidic chip to capture inflammatory monocytes as a diagnostic agent for assessing cardiovascular health. The apparatus and methods can rate disease progression and facilitate treatments and management of patients with acute coronary syndrome.

### 2. Background Discussion

Cardiovascular disease is the leading cause of morbidity and mortality in the United States as well as in developing nations. Several measurable factors that are associated with the risk of developing cardiovascular diseases include: low density lipoprotein cholesterol levels, triglyceride levels, and obesity. However, these measurements fail to identify as many as a third of the individuals that will eventually develop cardiovascular disease. They also do not report on the extent of disease progression during acute coronary syndrome. Conventional clinical assessments and methods for predicting risk are normally based on the results of electrocardiography and markers indicating injury. However, these assessments are not sufficiently accurate for predicting future risk of clinical outcomes for patients with ACS including acute in-stent thrombosis, in-stent restenosis requiring re-intervention, and myocardial infarction.

A large portion of the deaths related to cardiovascular diseases each year is due to inflammatory mediated coronary artery disease such as atherosclerosis. Conventional factors including smoking, diabetes, hypertension, C-reactive protein, and dyslipidemia that can provide some value in predicting overall risk in the population. However, a large percentage of patients either exhibit only one factor or do not exhibit any of these factors. In patients with coronary artery disease, there are presently no diagnostic procedures that can accurately identify those patients with a high probability of coronary complications during and post interventional therapy.

Inflammatory white blood cells in the circulatory system are intimately involved in the initiation and progression of cardiovascular diseases and have recently emerged as potential biomarkers for cardiovascular disease risk and outcome after interventional therapy. Cardiologists perform an average of 600,000 percutaneous coronary interventional procedures annually. However, the recurrence of acute coronary syndromes after 3 years post treatment is approximately 28%, resulting in about 200,000 patients returning to the clinic for additional treatment.

To predict patients who experience coronary complications, clinicians often rely on molecular biomarkers in circulating blood such as C-reactive protein (CRP), troponin, and the N-terminal of the pro-hormone brain natriuretic peptide (nt-proBNP). However, these biomarkers are not accurate at identifying those patients at highest risk of secondary coronary events. Therefore there is a need for the discovery of new biomarkers that provide a more dynamic measure of ongoing cardiovascular disease based upon the extent of inflammation with a goal of providing personalized data to guide clinicians in prescribing more intense lifestyle modification and/or therapy.

Accordingly, there is a need for diagnostic systems with predictive biomarkers that can facilitate early detection of cardiovascular diseases and guide risk factor modification and patient therapy following treatment.

The present technology satisfies this need and is generally an improvement in the art.

### BRIEF SUMMARY

Myocardial infarction remains one of the leading causes of mortality and morbidity and involves a high cost of care. The classification of patient conditions into low, intermediate, and high risk of a cardiac event including death is valuable in deciding on the appropriate course of treatment for the patient. Early prediction can be helpful in preventing the development of myocardial infarction with appropriate diagnosis and treatment. The present system uses predictive biomarkers that facilitate early detection of cardiovascular disease and can guide risk factor modification and therapy following treatment.

A diagnostic system for assessing cardiovascular health is provided that incorporates a microfluidic platform and sensors, denoted an artery-on-a-chip (or A-Chip). The microfluidic platform shears white cells in a small volume of blood (˜50 μL) over a glass substrate that mimics the molecular substrate and shear stress of an inflamed artery. The A-Chip captures an inflammatory subset of activated white blood cells that play a critical role in progression of cardiovascular disease and whose numbers in blood and efficiency of capture directly correlate with risk and pathogenesis of cardiovascular disease.

In particular, the assay examines the recruitment capacity of inflammatory monocytes, a process that is closely connected with the disease pathogenesis. Atherogenesis is associated with the persistent recruitment of circulating monocytes to inflamed endothelium expressing vascular cell adhesion molecule-1 (VCAM-1). The beta1-integrin very late antigen-4 (VLA-4) is the primary monocyte receptor that binds to VCAM-1 and supports cell rolling and shear stress resistant arrest during recruitment from the blood stream. The extent of monocyte activation in a whole blood sample can be measured by shearing it along a VCAM-1 molecular sensor and enumerating the number of monocytes captured relative to their concentration in blood. A direct correlation has been shown to exist between the number of inflammatory monocytes captured on the A-chip and coronary artery disease risk in subjects with dyslipidemia and obesity. Moreover, monocyte capture was shown to be significantly greater in patients being treated in the catheterization clinic and increased in proportion to established markers of the severity of coronary injury (e.g. creatine kinase and troponin).

There are several factors that contribute to cardiovascular disease like high LDL, TGRL, and cholesterol rich lipoproteins, infections, genetics, etc. These factors can also be considered in conjunction with results of the assay of the recruitment capacity of inflammatory monocytes to evaluate the cardiovascular risk and incidence of myocardial injury in patients.

The device is preferably a microfluidic sensor platform and readout that can be used in cardiac catheterization labs for point of care preclinical evaluations. In one embodiment, the A-chip unit has an input for receiving blood that has been extracted from the patient, a sensor and an output. The assay uses only a few droplets of blood while current lab tests use a few milliliters. The blood from the input flows through a microfluidic channel to a receptor laden sensor. In order to capture inflammatory monocytes, whole blood is sheared over VCAM-1 at a flow rate of about 12 μl/min, which will produce a wall shear stress of approximately 2 dynes/cm². The monocytes captured on the sensor may be distinguished by two-color fluorescence detection using antibodies to CD14 and CD16, respectively, that has been added to the blood prior to assay, in this embodiment.

A constant flow rate over the sensor can be achieved by applying a constant vacuum at the outlet. The vacuum pressure needed to drive the flow at a rate of 12 μl/min over the sensor is only about 44 Pa, which cannot be induced with a high degree of accuracy. Therefore, a resistance channel to the flow line may be added to increase the required vacuum pressure at the outlet. The pressure drop in the resistance channel is a function of friction factor, density, channel dimensions, and velocity. Friction factor (f) is a function of channel aspect ratio and Reynolds number and can be calculated from the Shah and London correlation.

In a further embodiment, the flow of blood is maintained without the use of a mechanical pump to create a negative pressure. An evacuated container of selected size is used as the vacuum source to produce a defined magnitude of negative pressure by connecting it to a box at atmospheric pressure. The vacuum pressure can be adjusted by changing the box volume. Blood from an input is drawn across the sensor that has one or more types of molecules that provides a ligand for VLA-4 and a monoclonal antibody that targets the high affinity conformation of the CD11c component of the integrin complex used by monocytes to adhere to inflamed arteries, for example.

The system may be self contained with A-chip, optical sensors, optional pumps, reagents, computer processor or controller, memory and interface/readout. The blood sample is injected in a port for analysis and the results are processed by the controller and displayed or stored.

In an alternative embodiment, the A-chip's molecular sensor, that discriminates activated inflammatory monocytes from whole blood, can be housed in a portable cassette for automated enumeration. For example, a commercial A-chip cartridge may include metering and mixing chambers and blister packs filled with reagents to be used in the dilution and washing steps of the assay. The A-chip reader includes an automated optical detection subsystem to enumerate inflammatory monocytes captured on the sensor. The flow delivery subsystem integrated in the device will include actuators and a small precision pump to generate the required vacuum. Clinicians will be able to print the test results through the user interface.

According to one aspect of the technology, a diagnostic system is provided with a structure that captures an inflammatory subset of activated white blood cells that play a critical role in the progression of cardiovascular disease and whose numbers in blood and efficiency of capture directly correlate with risk and pathogenesis of cardiovascular disease.

Another aspect of the technology is to provide an inexpensive, pumpless, portable, operator friendly A-chip unit that can be used in cardiac catheterization labs for point of care preclinical examinations.

A further aspect of the technology is to provide a diagnostic system that will allow the classification of patient conditions into low, intermediate, and high risk of cardiac events and allow the appropriate course of treatment for the patient.

Another aspect of the technology is to provide a system that is inexpensive to run and an assay that is significantly cheaper to run and provides a rapid readout than other assays that assess cardiovascular health (e.g. high sensitivity tropnin, nt-proBNP).

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a schematic perspective view of a microfluidic sensor chip (A-chip) element of the diagnostic system according to one embodiment of the technology.

FIG. 2 is a schematic diagram of a pumpless A-chip element of the diagnostic system according to an alternative embodiment of the technology.

FIG. 3 is a schematic perspective view of one an A-chip reader with cassette, computer processor and readout.

FIG. 4 is a schematic cross-section of an artery showing the persistent recruitment of circulating monocytes with inflamed endothelium expressing vascular cell adhesion molecule-1 (VCAM-1) including monocyte activation, adherence and plaque formation.

FIG. 5A is a schematic cross-section of the sensor of the A-chip showing the primed Mon2 capture on anti-CD11c.

FIG. 5B is a schematic cross-section of the sensor of the A-chip showing the captured and activated Mon2 that has been captured on anti-CD11c and arrested via high avidity VLA-4 according to one embodiment of the technology.

FIG. 6A is a graph of percentage of monocyte recruitment demonstrating low risk, high risk and myocardial infarction.

FIG. 6B is a graph showing the percentage of Mon2 recruitment as a function of Mon2 CD11c expression. Mon2 captured on the sensor is in direct proportion to a patient's CAD status.

### DETAILED DESCRIPTION

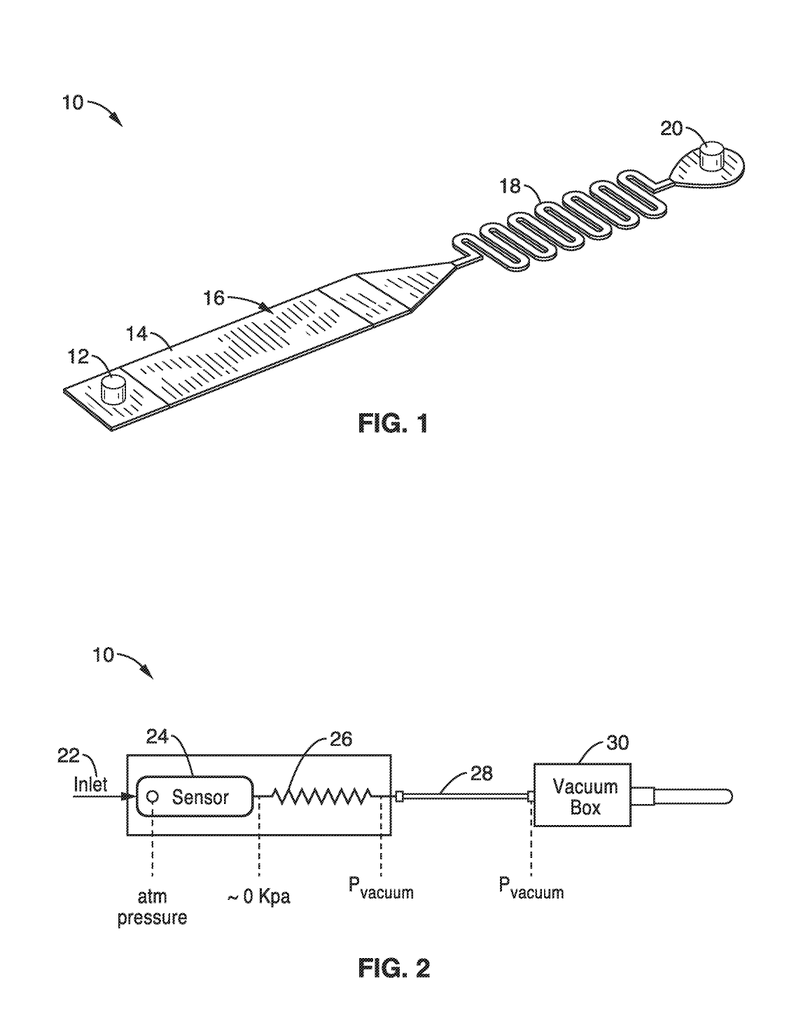

Referring more specifically to the drawings, for illustrative purposes, embodiments of the methods and resulting structures are generally shown. Several embodiments of the technology are described generally in FIG. 1 through FIG. 6B to illustrate the system, devices and methods. It will be appreciated that the methods may vary as to the specific steps and sequence and the system and devices may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, one preferred embodiment 10 of a microfluidic sensor chip structure 10 of the diagnostic system according to the technology is generally shown to illustrate one suitable structure. In this embodiment of the A-chip element of the system, the microfluidic chip 10 has an input port 12 for the introduction of the blood sample as well as an optional carrier fluid, washing fluids and fluorescent markers for labeling the captured monocytes. The input port 12 is connected to an enclosed sensor 14 array that has suitable active materials 16 on a surface that is preferably enclosed within a sensor housing in the microfluidic network. The enclosed chamber with the sensor surface with the active materials 16 is joined to an optionally serpentine shaped flow channel 18 and an output port 20. A vacuum source such as a pump is coupled to the output port 20 so that a controlled flow through the sensor chip is accomplished from the input port 12, across the sensor 14, through the flow channel 18 and out of the output port 20.

The flow rate across the sensor and the vacuum pressure that is required at the output port can be controlled by the selection of the dimensions of the flow line 18 and the associated resistance produced by the line. The pressure drop in the resistance channel 18 is a function of friction factor, density, channel dimensions, and velocity. Friction factor (f) is a function of channel aspect ratio and Reynolds number and can be calculated from Shah and London correlation.

The flow rate can be optimized for the selected dimensions of sensor to provide shear conditions for capturing activated monocytes by the sensor active material. The preferred flow rate over the sensor is approximately 12 μl/min that will produce a wall shear stress of about 2 dynes/cm². Because the vacuum pressure required to produce a flow rate of 12 μl/min over the sensor is only 44 Pa, which cannot be induced with a high degree of accuracy, a resistance channel 18 may be used.

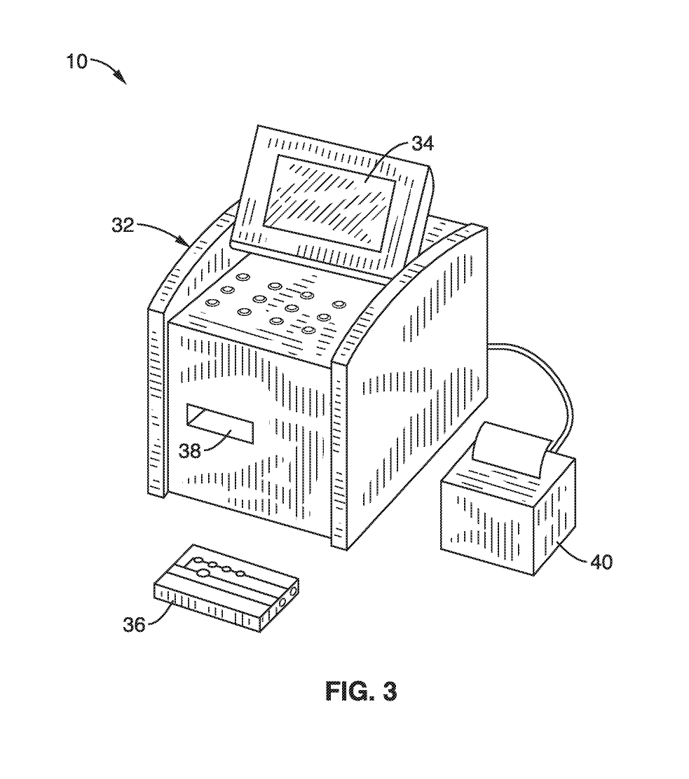

An alternative embodiment of the A-chip element is shown schematically in FIG. 2 that does not require a pump. The A-chip element 10 in this embodiment has an inlet 22 for the introduction of the blood sample and carrier fluids. The fluid flow can also be modulated by a resistance channel 26 attached to the flow line 28 to increase the required vacuum pressure. A constant flow rate over the sensor can be achieved by applying a constant vacuum at the outlet provided by a use of an evacuated container or chamber 30 as the vacuum source. The vacuum pressure by adjusted by changing the volume of the evacuated box 30.

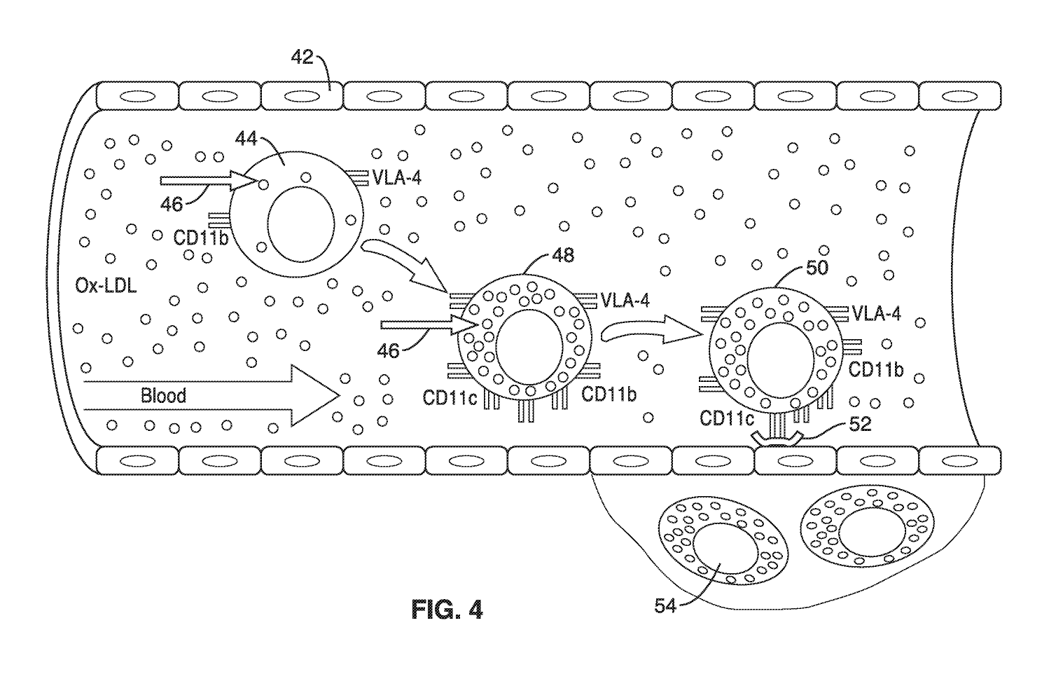

The A-chip can be incorporated into a cassette 36 that is inserted into a slot 38 of a reader 32 as shown in the embodiment of FIG. 3. Here the reader 32 has a detector, a computer processor with an interface and a display 34 along with a printer readout 40. In one embodiment, the cassette structure includes an evacuated chamber or container and is self contained. The reader 32 reads the results of the flow. In another embodiment, the reader includes the pumps and carrier fluids and the sample shear flow takes place in the reader. The binding of monocytes to the sensor surface is thereafter detected and analyzed. Although optical detection using an immunofluorescence scheme is preferred, other methods for quantifying the number of monocytes bound can be used such as electronic means.

The computer of reader 32 has programming that can process data received from the sensor and dynamically measure ongoing cardiovascular disease based upon the extent of inflammation with a goal of providing personalized data to guide clinicians in prescribing more intense lifestyle modification and/or therapy. The computer programming of reader 32 can also account for the results of other tests performed on a particular patient in the formulation of an assessment of the risk of future cardiac events for that patient. Conventional factors such as smoking, diabetes, hypertension, C-reactive protein, and dyslipidemia and the health history of the patient can also be considered in the identification of patients with a high probability of coronary complications during and post interventional therapy.

For example, within minutes the assay can provide a rapid measure of monocyte activation state that can resolve differences between subjects with risk factors for atherosclerosis or with established coronary disease such as acute MI. In the case of early atherosclerosis, identification of Mon2 activation may prompt a clinician to prescribe more intensive lifestyle modifications that are specific to predisposing risk factors, such as dietary lipids or high blood pressure. Among patients with MI, increased Mon2-CD11c activation may identify patients at elevated risk for recurrent cardiovascular events. Specific interventions to abrogate Mon2-CD11c activation could also limit infarct size by limiting Mon2 localization during an acute MI.

The molecular sensor 14 of the A-chip separates activated inflammatory monocytes from whole blood. It has been shown that the number of activated monocytes that arrest in the shear flow increases in proportion to a patient's coronary artery disease status. Since atherogenesis is associated with monocyte recruitment to arterial vessels expressing VCAM-1, the elevated monocyte arrest on an arterial mimetic sensor is predictive of the extent of coronary disease of a subject and an indicator of negative clinical outcomes in coronary artery disease patients.

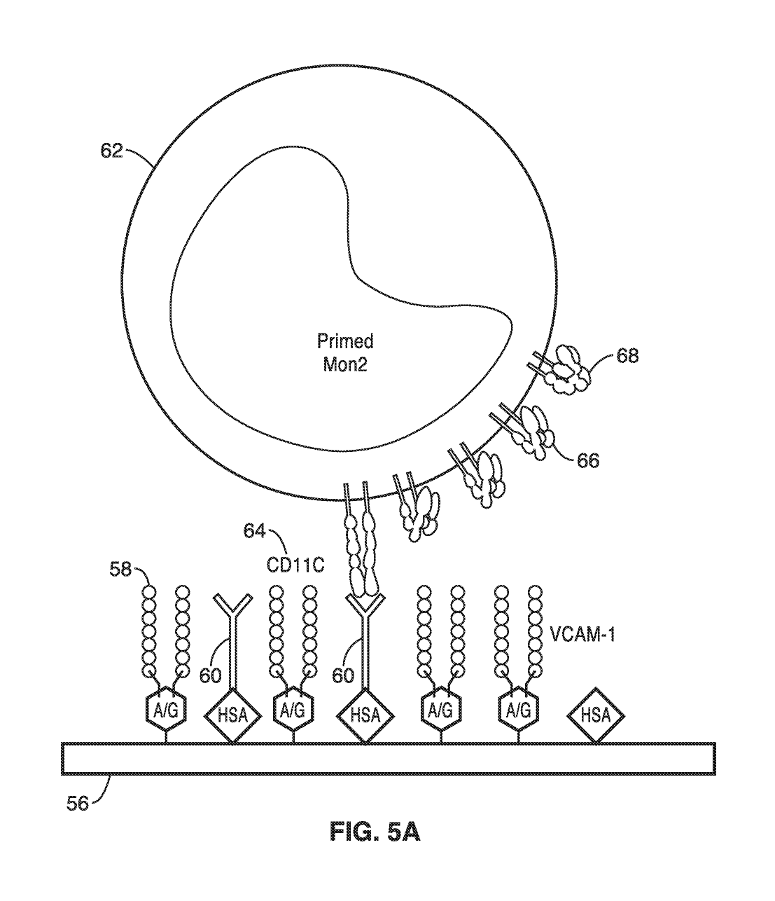

Key events in the development of atherosclerosis are the activation and migration of monocytes into the arterial wall. This recruitment process is shown generally in the cross-section of an artery 42 in FIG. 4. Three distinct monocyte subsets are detected in circulation: (classical CD14++CD16−, Mon1), (intermediate CD14++CD16+, Mon2) and (non-classical CD14+CD16++, Mon3), and each subset may play distinct roles during atherogenesis and myocardial infarction.

In the process illustrated schematically in FIG. 4, the Mon2 monocyte 44 has down regulated the CD11c (i.e. CD11⁻) and there are few scavenger receptors 46 of triglycerides or triglyceride-rich lipoproteins (TGRLs).

In response to a high-fat meal, CD11c expression on the Mon2 monocyte 48 increased significantly, whereas expression of other adhesion receptors did not change significantly on Mon2 or other subsets. Furthermore, CD11c was observed to be upregulated 300% on patients undergoing myocardial infarction compared to healthy subjects. There was also a trend for increased CD11c with triglycerides 46 that reached significance at levels above 200 mg/dL only on Mon2, whereas CD11c expression on the other subsets do not change after eating.

Adhesion of an activated Mon2 monocyte 50 is also shown in FIG. 4. Atherogenesis is associated with the persistent recruitment of circulating monocytes 50 to inflamed endothelium expressing vascular cell adhesion molecule-1 (VCAM-1) 52. The β₁-integrin called very late antigen-4 (VLA-4) is the primary monocyte receptor that binds to VCAM-1 and supports cell rolling and arrest during recruitment from the blood stream. The β₂-integrin CD11c/CD18 also supports monocyte capture on VCAM-1 and can activate VLA-4 to bind VCAM-1 as well.

In addition, CD11c is a reliable biomarker of diet-induced monocyte activation, since dyslipidemic humans and those with coronary artery disease upregulate CD11c expression on an inflamed subset of monocytes (e.g. Mon2; CD14⁺⁺CD16⁺) in the circulation. Monocyte CD11c is also a potential therapeutic target for ameliorating atherosclerosis, since genetic deletion of CD11c in hypercholesterolemic mice (e.g. apoE^{-/-}) results in smaller atherosclerotic lesions with decreased macrophage content.

After adhesion, the monocytes cross over the endothelial barrier and out of the artery. After crossing over, the monocytes 54 differentiate into macrophages that perpetuate the inflammatory response.

The device is designed to gauge the inflammatory status of a patient by measuring monocyte function from a minute blood sample (<0.1 mL) using a single use disposable rapid readout device. The A-chip is primed by placing whole blood in the reservoir where it is sheared across the sensor, which is designed to specifically capture inflammatory monocytes (e.g. Mon2; CD14⁺⁺CD16⁺).

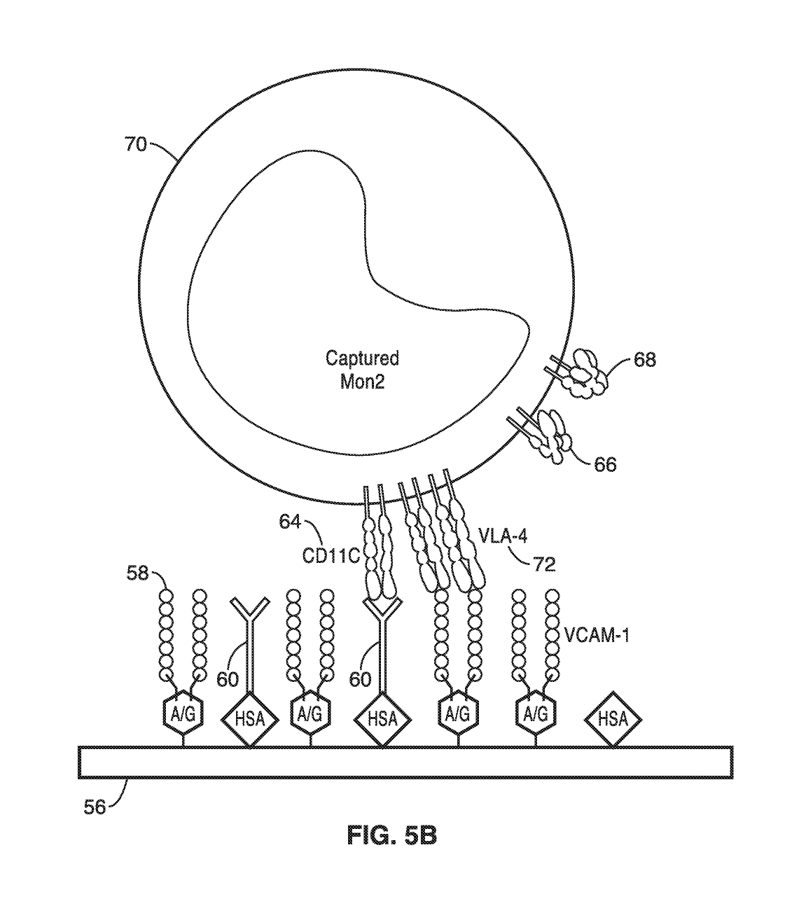

The arrest of primed or activated monocytes on the sensor surface is shown in FIG. 5A and FIG. 5B. Monocytes from humans with coronary artery disease are induced to capture on the A-chip sensor through a similar mechanism involving activation of VLA-4 to bind to its ligand VCAM-1 as well. Monocyte capture on the sensor active materials (anti-CD11c and VCAM-1) is regulated by the state of activation of CD11c and VLA-4.

In the embodiment of the microfluidic sensor active surface shown in FIG. 5A, the sensor surface 56 has been functionalized with vascular cell adhesion molecule-1 (VCAM-1) molecules 58 and anti-CD11c antibodies 60. The allosteric antibody 60 that binds activated CD11c receptor 64 on primed inflammatory monocytes 62 initiates their capture as seen in FIG. 5A.

The CD11c activation state regulates the amount of VLA-4 enrichment that can interact with the contact region of the sensor to support the shear-resistant monocyte capture on the sensor surface. The CD11c and VLA-4 cooperate in mediating the arrest of primed monocytes 70 on sensor surface active materials (VCAM-1 and anti-CD11c) as shown in FIG. 5B.

This process is specific to the Mon2 subset, in that the VLA-4 72 is co-localized with the bound CD11c so that the high avidity receptors 72 are brought into a position to bind VCAM-1 58 that is co-presented with anti-CD11c on the sensor surface. Using this approach, the frequency of capture of monocytes 70 in blood directly correlates with the severity of coronary artery disease ranging from stable angina to myocardial infarction. Other CD11c molecules 66 and VLA-4 molecules 68 on the surface may also participate in the binding of the monocyte if the monocyte rolls in the fluid flow so that the VLA-4 molecules 68 or CD11c molecules 68 are in proximity to their corresponding receptors or antibodies.

The invention may be better understood with reference to the accompanying examples, which are intended for purposes of illustration only and should not be construed as in any sense limiting the scope of the present invention as defined in the claims appended hereto.

### Example 1

In order to prove the concept of the device and the fabrication methods, a microfluidic sensor device was produced and tested. Devices were designed to allow utility of four independent flow channels with dimensions of 60 μm×2 mm×8 mm (h×w×l). A spider web of interconnected vacuum channels permitted the device to be reversibly vacuum-sealed to a functionalized glass cover slip. The microfluidic networks were designed in AutoCAD (Autodesk) and then printed as a photomask by CAD/Art Services Inc. Master molds for the devices were fabricated by patterning SU-8 50 photoresist (MicroChem) on a 200 mm diameter silicon wafer to a height of 60 μm and exposing the photopolymer to UV light through the photomask containing the design of the microfluidic network as described previously. By casting polydimethylsiloxane (PDMS) Sylgard 184 prepolymer (Dow Corning) over the masters, PDMS replicas were produced. Flow and vacuum access holes were punched directly into PDMS replicas.

An acrylic platform was also produced to reduce the pressure of the 3 mL Vacutainer® such that the resulting pressure differential between the inlet and outlet of the A-Chip is −2 kPa. It is created using acrylic (TAP Plastics), Tygon tubing (McMaster), and acrylic cement (TAP Plastics).

Pieces of acrylic were cut out from a stock piece using a high-powered laser cutter. The pieces were then assembled and bonded using acrylic cement. The final dimensions of the platform are 6 cm×8 cm×2 cm (l×w×h). Inlet and outlet holes were drilled and then tapped into the acrylic platform. The inlet hole was connected to the A-Chip via Tygon tubing and a 21-gauge blunted needle. The outlet hole was configured to connect to a 3 mL Vacutainer®.

The inside of the A-Chip was coated with Vascular Cell Adhesion Molecule-1 (VCAM-1) at 10 μg/mL concentration overnight at 4° C. Following the VCAM-1 coating, the A-Chip was then placed on the platform and the Tygon tubing was connected. A 50 μL sample of blood (diluted 1:5 with HBSS+Ca/Mg (LifeTechnologies)) was added to the reservoir. A 3 mL Vacutainer was then connected to the platform that initiated the blood flow through the A-Chip.

### Example 2

The devices were tested to gauge the inflammatory status by measuring monocyte function from a minute blood sample (<0.1 mL) The A-chip was primed by placing whole blood in the reservoir where it was sheared across the sensor, which was designed to specifically capture inflammatory monocytes (e.g. Mon2; CD14⁺⁺CD16⁺). An allosteric antibody that binds activated CD11c receptor on primed inflammatory monocytes initiated their capture. This process was specific to the Mon2 subset, in that VLA-4 is co-localized with CD11c in a process that brings high avidity receptors proximal to bind VCAM-1 co-presented with anti-CD11c on the sensor. Using this approach, it was shown that that the frequency of capture of monocytes in blood directly correlates with the severity of coronary artery disease ranging from stable angina to myocardial infarction.

Preliminary data indicated that inflammatory monocytes identified by CD14⁺⁺CD16⁺ expressing Mon2 on chip were captured at ˜50-fold higher frequency than other leukocytes based upon upregulation of CD11c expression and activated VLA-4 that binds VCAM-1 which is chemically linked to the glass sensor substrate. An anti-CD11c monoclonal antibody (496B) was shown to enhance the detection by 10-fold when it was annealed to the glass along with VCAM-1. The mechanism for this amplification involves a cooperative process by which the two integrins become co-localized on imminently activated monocytes in circulation.

In essence, this antibody facilitates the capture of those monocytes most susceptible to integrin induced inflammatory activation and capture on VCAM-1 on the A-chip sensor. Automated fluorescence discrimination was then used to count the number of inflammatory monocytes on the sensor.

### Example 3

To demonstrate the measurement of the extent of monocyte activation in a whole blood sample by shearing it along a VCAM-1 molecular sensor and enumerating monocyte capture, many different samples were processed and the results evaluated in view of known conditions for the subject patients. The number of Mon2 cells captured on the sensor is in direct proportion to a patient's CAD status.

The β₁ and β₂-integrins CD11c/CD18 and CD49d/CD29 (VLA-4) were shown to be involved in recruitment of inflammatory monocytes on pro-atherogenic arterial endothelium. A direct correlation between the number of inflammatory monocytes captured on the A-chip and coronary artery disease risk in subjects with dyslipidemia and obesity. Moreover, monocyte capture was significantly greater in patients being treated in the catheterization clinic and increased in proportion to established markers of the severity of coronary injury (e.g. creatine kinase and troponin).

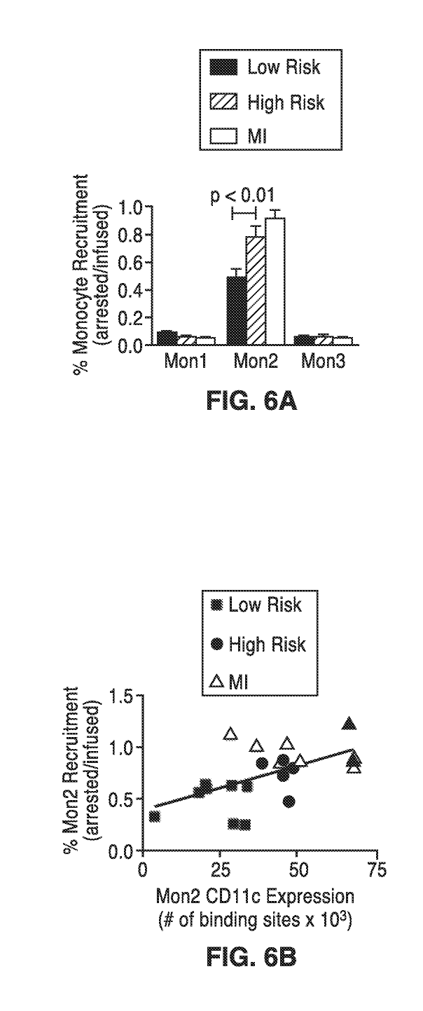

Postprandial monocyte recruitment efficiency for subjects categorized as low (fTG <200 mg/dL, n=8) and high risk (fTG >200 mg/dL, n=5) and MI patients (n=8) is shown in FIG. 6A. Recruitment efficiency plotted as a function of CD11c expression for postprandial study subjects and MI patients is shown in FIG. 6B. A regression curve was fit to the means of low-risk, high-risk, and MI groups (Pearson r=0.9957, R2=0.9915, P=0.0587).

From the description herein, it will be appreciated that that the present disclosure encompasses multiple embodiments which include, but are not limited to, the following:

1. A microfluidic chip device for detecting activated monocytes from a sample of blood, comprising: (a) a microfluidic chip having a flow channel coupled to a sample inlet and an outlet; and (b) a sensor with an inner surface coated with anti-CD11c antibodies or VLA-4 receptor substrate, the sensor fluidly coupled to the flow channel, the sensor configured to shear white cells in a volume of blood flowing through the flow channel and the sensor; (c) wherein activated monocytes from a sample of blood adhere to the inner surface of the sensor.

2. The device of any preceding embodiment, wherein the inner surface of the sensor is coated with anti-CD11c antibodies and VLA-4 receptor substrate.

3. The device of any preceding embodiment, wherein the VLA-4 receptor substrate comparises vascular cell adhesion molecule-1 (VCAM-1).

4. The device of any preceding embodiment, further comprising: a resistance channel coupled to the flow channel on one end and the output port on the other end; wherein a negative pressure that is required to be applied to the flow channel to cause fluid to flow can be controlled by the dimensions of the resistance channel.

5. The device of any preceding embodiment, further comprising: an evacuated container coupled to the output port of the chip; wherein a pressure differential occurs between the inlet and the outlet; and wherein fluid flows between the inlet and the outlet without the use of a pump.

6. The device of any preceding embodiment, further comprising: a fluid reservoir coupled to the flow channel; wherein fluid flowing through the flow channel and sensor flows at a controlled flow rate.

7. The device of any preceding embodiment, wherein a fluid flow through the sensor creates a wall shear stress of about 2 dynes/cm.²

8. The device of any preceding embodiment, wherein a sample of whole blood is sheared over anti-CD11c antibodies or VLA-4 receptor substrate at a flow rate of about 12 μl/min.

9. A microfluidic apparatus for detecting activated monocytes from a sample of blood, comprising: (a) a system of microfluidic flow channels coupled to a sample input port and an output port; (b) a sensor fluidly coupled to the flow channels, the sensor having an inner surface coated with anti-CD11c antibodies or VLA-4 receptor substrate configured to specifically capture inflammatory monocytes; and (c) a detector associated with the sensor; (d) wherein capture of monocytes from a sample by the sensor is detected by the detector and quantified.

10. The apparatus of any preceding embodiment, further comprising at least one fluorescent label and an optical detector.

11. The apparatus of any preceding embodiment, wherein the fluorescent label comprises two-color fluorescence detection using antibodies to CD14 and CD16.

12. The apparatus of any preceding embodiment, wherein the inner surface of the sensor is coated with anti-CD11c antibodies and VLA-4 receptor substrate.

13. The apparatus of any preceding embodiment, further comprising a vacuum source coupled to the output port of the flow channels of the microfluidic system.

14. The apparatus of any preceding embodiment, wherein the vacuum source comprises an evacuated container; and wherein a pressure differential occurs between the inlet and the outlet without the use of a pump.

15. The apparatus of any preceding embodiment, further comprising a resistance channel coupled to the output port and flow channels configured to control the flow rate of fluid across the sensor.

16. A system for detecting activated monocytes from a sample of blood, comprising: (a) a sample cassette, the cassette comprising (i) a microfluidic chip having a sample inlet and an outlet and a flow channel therebetween; and (ii) a sensor with an inner surface coated with anti-CD11c antibodies and VLA-4 receptor substrate, the sensor fluidly coupled to the flow channel, the sensor configured to shear white cells in a volume of blood flowing through the flow channel and the sensor; (b) a detector, the detector comprising: (i) a vacuum source configured to couple with the output port of the microfluidic chip; (ii) a source of fluid configured to couple with the input port; (iii) a detector associated with the sensor capable of detecting and quantifying monocytes bound to the sensor.

17. The system of any preceding embodiment, wherein the microfluidic chip further comprises a resistance channel coupled to the output port and flow channel configured to control the flow rate of fluid across the sensor.

18. The system of any preceding embodiment, wherein the sample cassette further comprises a vacuum source comprising an evacuated container; wherein a pressure differential occurs between the inlet and the outlet without the use of a pump.

19. The system of any preceding embodiment, wherein the detector comprises an optical detector configured to detect monocytes labeled with at least one type of fluorescent label.

20. The system of any preceding embodiment, further comprising: a computer processor operably coupled to the detector and vacuum source mechanisms of the detection platform; and a non-transitory computer-readable memory storing instructions executable by the computer processor; wherein the instructions, when executed by the computer processor, perform steps comprising: capturing CD11c receptor activated monocytes from a sample of blood; measuring the number of activated monocytes captured from the sample; and rating risk based on measured activated monocytes.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, computer processor, central processing unit (CPU), and computer are used synonymously to denote a device capable of executing the instructions and communicating with input/output interfaces and/or peripheral devices, and that the terms processor, computer processor, CPU, and computer are intended to encompass single or multiple devices, single core and multicore devices, and variations thereof.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A microfluidic chip device for detecting activated monocytes from a sample of blood, comprising:
(a) a microfluidic chip having a flow channel coupled to a sample inlet and an outlet; and
(b) a sensor with an inner surface coated with anti-CD11c antibodies or VLA-4 receptor substrate, the sensor fluidly coupled to the flow channel, the sensor configured to shear white cells in a volume of blood flowing through the flow channel and the sensor;
(c) wherein activated monocytes from a sample of blood adhere to the inner surface of the sensor.

2. The device of claim 1, wherein said inner surface of said sensor is coated with anti-CD11c antibodies and VLA-4 receptor substrate.

3. The device of claim 1, wherein said VLA-4 receptor substrate comparises vascular cell adhesion molecule-1 (VCAM-1).

4. The device of claim 1, further comprising:
a resistance channel coupled to the flow channel on one end and the output port on the other end;
wherein a negative pressure that is required to be applied to the flow channel to cause fluid to flow can be controlled by the dimensions of the resistance channel.

5. The device of claim 1, further comprising:
an evacuated container coupled to the output port of the chip;
wherein a pressure differential occurs between the inlet and the outlet; and
wherein fluid flows between the inlet and the outlet without the use of a pump.

6. The device of claim 1, further comprising:
a fluid reservoir coupled to the flow channel;
wherein fluid flowing through the flow channel and sensor flows at a controlled flow rate.

7. The device of claim 1, wherein a fluid flow through said sensor creates a wall shear stress of about 2 dynes/cm.²

8. The device of claim 1, wherein a sample of whole blood is sheared over anti-CD11c antibodies or VLA-4 receptor substrate at a flow rate of about 12 μl/min.

9. A microfluidic apparatus for detecting activated monocytes from a sample of blood, comprising:
(a) a system of microfluidic flow channels coupled to a sample input port and an output port;
(b) a sensor fluidly coupled to the flow channels, said sensor having an inner surface coated with anti-CD11c antibodies or VLA-4 receptor substrate configured to specifically capture inflammatory monocytes; and
(c) a detector associated with the sensor;
(d) wherein capture of monocytes from a sample by the sensor is detected by the detector and quantified.

10. The apparatus of claim 9, further comprising:
at least one fluorescent label; and
an optical detector.

11. The apparatus of claim 10, wherein said fluorescent label comprises two-color fluorescence detection using antibodies to CD14 and CD16.

12. The apparatus of claim 9, wherein said inner surface of said sensor is coated with anti-CD11c antibodies and VLA-4 receptor substrate.

13. The apparatus of claim 9, further comprising:
a vacuum source coupled to the output port of the flow channels of the microfluidic system.

14. The apparatus of claim 13, wherein the vacuum source comprises an evacuated container; and
wherein a pressure differential occurs between the inlet and the outlet without the use of a pump.

15. The apparatus of claim 9, further comprising a resistance channel coupled to the output port and flow channels configured to control the flow rate of fluid across the sensor.

16. A system for detecting activated monocytes from a sample of blood, comprising:
(a) a sample cassette, the cassette comprising:
(i) a microfluidic chip having a sample inlet and an outlet and a flow channel therebetween; and
(ii) a sensor with an inner surface coated with anti-CD11c antibodies and VLA-4 receptor substrate, the sensor fluidly coupled to the flow channel, the sensor configured to shear white cells in a volume of blood flowing through the flow channel and the sensor;
(b) a detection platform, the detection platform comprising:
(i) a vacuum source configured to couple with the output port of the microfluidic chip;
(ii) a source of fluid configured to couple with the input port;
(iii) a detector associated with the sensor capable of detecting and quantifying monocytes bound to the sensor.

17. The system of claim 16, wherein said microfluidic chip further comprises a resistance channel coupled to the output port and flow channel configured to control the flow rate of fluid across the sensor.

18. The system of claim 16, wherein said sample cassette further comprises a vacuum source comprising an evacuated container;
wherein a pressure differential occurs between the inlet and the outlet without the use of a pump.

19. The system of claim 16, wherein said detector comprises an optical detector configured to detect monocytes labeled with at least one type of fluorescent label.

20. The system of claim 16, further comprising:
a computer processor operably coupled to the detector and vacuum source mechanisms of the detection platform; and
a non-transitory computer-readable memory storing instructions executable by the computer processor;
wherein said instructions, when executed by the computer processor, perform steps comprising:
capturing CD11c receptor activated monocytes from a sample of blood;
measuring the number of activated monocytes captured from the sample; and
rating risk based on measured activated monocytes.
